(19) United States
(12) Patent Application Publication
(10) Pub. No.: US 2003/0171391 A1
(43) Pub. Date: Sep. 11, 2003

(54) Ambroxol for the treatment of chronic pain

(75) Inventors: Wolfram Gaida, Ingelheim (DE); Klaus Klinder, Oggelshausen (DE); Thomas Weiser, Wiesbaden (DE)

Correspondence Address: BOEHRINGER INGELHEIM CORPORATION, 900 RIDGEBURY ROAD, P. O. BOX 368, RIDGEFIELD, CT, 06877, US

(73) Assignee: Boehringer Ingelheim Pharma GmbH & Co. KG, Ingelheim (DE)

(21) Appl. No.: 10/348,283

(22) Filed: Jan. 21, 2003

Related U.S. Application Data
(60) Provisional application No. 60/385,874, filed on Jun. 5, 2002.

(30) Foreign Application Priority Data
Jan. 25, 2002 (DE) 102 03 104.5

(51) Int. Cl. A61K 31/60; A61K 31/485; A61K 31/192; A61K 31/415; A61K 31/195

(52) U.S. Cl. 514/282; 514/561; 514/165; 514/406; 514/570

(57) ABSTRACT

The invention relates to the use of ambroxol and the pharmacologically acceptable salts thereof for preparing a pharmaceutical composition for the treatment of diseases which are based on a powerful activation of voltage-dependent sodium channels, particularly for the treatment of chronic pain.

### RELATED APPLICATIONS

Benefit of U.S. Provisional Application Serial No. 60/385,874, filed on Jun. 5, 2002 is hereby claimed.

### FIELD OF THE INVENTION

The invention relates to the use of ambroxol and the pharmacologically acceptable salts thereof for the treatment of diseases which are based on powerful activation of voltage-dependent sodium channels, particularly for the treatment of chronic pain.

### BACKGROUND OF THE INVENTION

The active substance ambroxol (trans-4-(2-amino-3,5-dibrombenzylamino)-cyclohexanol) is a known antitussive and expectorant. In addition, the activity of ambroxol as a sodium channel blocker has been described in the literature (Society for Neuroscience Abstracts, 2000, Vol.26, No. 1-2).

The potential activity of sodium channel blockers as analgesics is also known from the prior art (Mao and Chen(2000), Pain 87, 7-17). However, known sodium channel blockers are unsuitable for the treatment of chronic pain and for treating diseases caused by excessive activation of voltage-dependent sodium channels, as they preferentially inhibit those sodium channels which play a minor part in the development and transmission of noxic signals in sensory neurones, i.e. those which may be inhibited by tetrodotoxin, in contrast to tetrodotoxin-resistant neuronal sodium channels (Rush and Elliott (1997), Neuroscience Letters 226, 95-98; Scholz et al. (1998); Journal of Neurophysiology 79, 1746-1754;Song et al. (1997), Journal of Pharmacology and Experimental Therapeutics, 282, 707-714).

Diseases connected with chronic or chronically recurrent pain include, inter alia, migraine, neuralgia, muscle pain and inflammatory pain. They share the same mechanisms as chronically recurrent pain [Dray, A. Urban L. and Dickenson, A. Trends in Pharmacological Sciences 1994; 15:190-197].

The chronic neuronal pains include inter alia postoperative pain, shingles, phantom pain, diabetic neuropathy, pain after chronic nerve compression as well as the final stages of Aids and cancer.

The aim of the present invention is to prepare an active substance for the treatment of diseases caused by excessively powerful activation of voltage-dependent sodium channels.

In particular, the aim of the present invention is to provide an active substance for the treatment of chronic pain, especially chronic neuronal or neuropathic pain, with good bioavailability and a strong antinociceptive activity.

### DESCRIPTION OF THE INVENTION

Surprisingly, ambroxol exhibits a very good activity in the treatment of chronic pain and neurological diseases, which is based on blocking excessively strongly activated voltage-dependent sodium channels, particularly excessively strongly activated voltage-dependent neuronal sodium channels.

The invention therefore relates to the use of ambroxol or one of the pharmacologically acceptable salts thereof for preparing a pharmaceutical composition for the treatment of diseases which are based on a powerful activation of voltage-dependent sodium channels.

Ambroxol or one of the pharmacologically acceptable salts thereof is preferably used to prepare a pharmaceutical composition for the treatment of chronic and/or neuropathic pain, preferably in diabetic neuropathy, postherpetic neuralgia, chronic back pain, migraine, trigeminal neuralgia or tumour pain, most preferably in diabetic neuropathy, postherpetic neuralgia, chronic back pain or migraine, most preferably in diabetic neuropathy or postherpetic neuralgia.

Most preferably, ambroxol or one of the pharmacologically acceptable salts thereof is used to prepare a pharmaceutical composition for the treatment of cerebral excitotoxicity-induced disorders, preferably epilepsy, brain trauma or cerebral stroke, more preferably epilepsy or brain trauma, most preferably epilepsy.

It is also particularly preferred to use ambroxol or one of the pharmacologically acceptable salts thereof to prepare a pharmaceutical composition for the treatment of cardiac arrhythmias.

The invention further relates to a pharmaceutical composition containing ambroxol and one or more active substances selected from among the anticonvulsants, for example barbiturates, preferably phenobarbital, hydantoins, succinimides, oxazolidines, benzodiazepines, neuroprotective substances, for example NMDA receptor antagonists, and antiarrhythmics, preferably lidocaine, verapamil or gallopamil.

It is particularly preferable to use ambroxol or one of the pharmacologically acceptable salts thereof in combination with one or more other active substances, selected from among the anticonvulsants, for example barbiturates, hydantoins, succinimides, oxazolidines, benzodiazepines, preferably phenobarbital, neuroprotective substances, for example NMDA receptor antagonists, or antiarrhythmics, preferably lidocaine, verapamil or gallopamil.

The invention further relates to a pharmaceutical composition containing ambroxol and one or more active substances selected from among the opiates, preferably morphine, buprenorphine, levomethadone, codeine, tramadol or tilidine, non-steroidal analgesics, for example acetylsalicylic acid, paracetamol, diclofenac, meloxicam, ibuprofen, ibuprofen lysinate, ibuprofen in extruded form (as described in WO 99/06038), gabapentine and antidepressants, preferably imipramine, maprotiline, mianserine, fluoxetine, viloxazine, tranylcypromine or moclobemide, and alpha-adrenergic agonists such as clonidine, for example.

It is also particularly preferred to use ambroxol or one of the pharmacologically acceptable salts thereof in combination with one or more other pain relievers selected from among the opiates, preferably morphine, buprenorphine, levomethadone, codeine, tramadol or tilidine, non-steroidal analgesics, for example acetylsalicylic acid, paracetamol, diclofenac, meloxicam, ibuprofen, ibuprofen lysinate, ibuprofen in extruded form (as described in WO 99/06038), gabapentine or antidepressants, preferably imipramine, maprotiline, mianserine, fluoxetine, viloxazine, tranylcypromine or moclobemide.

It is also preferable to use ambroxol for the treatment of patients suffering patient or patients with tumoral diseases.

Suitable acids for forming salts of ambroxol are for example hydrochloric acid, hydrobromic acid, sulphuric acid, phosphoric acid, nitric acid, oxalic acid, malonic acid, fumaric acid, maleic acid, tartaric acid, citric acid, ascorbic acid and methanesulphonic acid, preferably hydrochloric acid.

The activity of ambroxol according to the invention is to be illustrated by the following Examples. These are intended solely as an illustration of the invention and are not to be regarded as limiting.

Ambroxol exhibits an antinociceptive activity which is based on the blocking of voltage-dependent sodium channels. In contrast to the sodium channel blockers described which are used clinically, ambroxol preferentially inhibits tetrodotoxin-resistant sodium channels in nociceptive C-fibre neurones. Their special relevance to inflammatory and chronic pain has been demonstrated in vivo (Waxman et al. (1999) Proceedings of the National Academy of Science 96, 7635-7639; Khasar et al. (1998), Neuroscience Letters 256, 17-20).

In neurone cultures from the posterior root ganglions of adult rats, tetrodotoxin-resistant sodium channels were semi-maximally inhibited by 35 μM of ambroxol. Tetrodotoxin-sensitive currents were inhibited much less strongly by this concentration and here the IC₅₀ was higher than 100 μM.

The powerful analgesic effect of blocking the channels was demonstrated inter alia by animal experimentation using the formalin paw test on rats. The test is described below.

Formalin Paw Test (Ref: Carlton S M and Zhou S: Attenuation of formalin-induced nociceptive behaviors following local peripheral injection of gabapeptin. Pain 76, 201-207, 1998).

Male rats (Chbb: THOM) weighing 250-300 grams are used. 20 μl of a 2% formaldehyde solution is injected into the plantar region of the right hind paw. Immediately after, the number of flinches (twitches of the affected hind paw) and the length of time spent licking the affected paw are recorded over a period of one hour. After 5 minutes in each case the values are grouped together into epochs. From the epoch values, time-effect curves for flinches and licking are plotted. Typically, two phases of formalin effect are observed (flinches, licking). A first phase lasting 0-10 minutes and a second phase lasting 10-60 minutes. Between the two phases the number of flinches and duration of licking fall towards 0 (interphase). From the time-effect curves the areas under the curve for the first phase and for the second phase are determined. Usually, 5 animals are used for each control, placebo and dose of substance. The results of the doses of substance are compared with those of the controls and ED₅₀ values are calculated. The ED₅₀ is the dose at which the control values are inhibited by 50%.

The ED₅₀ value for ambroxol hydrochloride is 70 mg/kg p.o.

In other test models for neuropathic pain in the rat^{(1),(2)} ambroxol reduced the tactile allodynia, as well as the thermal hyperalgesia. ⁽¹⁾ Bennett G J and Xie Y-K. A peripheral mononeuropathy in rat that produces disorders of pain sensation like those seen in man. Pain 33, 87-107, 19883. ⁽²⁾ Seltzer, Z., Dubner R. and Shir, Y. A novel behavioral model of neuropathic pain disorder. Pain 43, 205-218, 1990.

Ambroxol may be used on its own or in conjunction with other pharmacologically active substances. Suitable preparations include for example tablets, capsules, suppositories, solutions, elixirs, emulsions or dispersible powders. Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanillin or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may for example be prepared by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

A therapeutically effective daily dose is between 30 and 4000 mg, preferably 100 to 2000 mg in adults.

The Examples which follow illustrate the present invention without restricting its scope:

### EXAMPLES OF PHARMACEUTICAL FORMULATIONS

| A) | Tablets | per tablet |
|---|---|---|
| | active substance | 800 mg |
| | lactose | 140 mg |
| | corn starch | 240 mg |
| | polyvinylpyrrolidone | 20 mg |
| | magnesium stearate | 10 mg |

Ambroxol, lactose and some of the corn starch are mixed together. The mixture is screened, then moistened with a solution of polyvinylpyrrolidone in water, kneaded, wet-granulated and dried. The granules, the remaining corn starch and the magnesium stearate are screened and mixed together. The mixture is compressed to produce tablets of suitable shape and size.

| B) | Tablets | per tablet |
|---|---|---|
| | ambroxol | 800 mg |
| | corn starch | 190 mg |
| | lactose | 55 mg |
| | microcrystalline cellulose | 35 mg |
| | polyvinylpyrrolidone | 20 mg |
| | sodium-carboxymethyl starch | 30 mg |
| | magnesium stearate | 10 mg |

The ambroxol, some of the corn starch, lactose, microcrystalline cellulose and polyvinylpyrrolidone are mixed together, the mixture is screened and worked with the remaining corn starch and water to form a granulate which is dried and screened. The sodium-carboxymethyl starch and the magnesium stearate are added and mixed in and the mixture is compressed to form tablets of a suitable size.

| C) | Coated tablets | per coated tablet |
|---|---|---|
| | Ambroxol | 500 mg |
| | Corn starch | 45 mg |
| | Lactose | 30 mg |
| | Polyvinylpyrrolidone | 5 mg |
| | Magnesium stearate | 5 mg |

The ambroxol, corn starch, lactose and polyvinylpyrrolidone are thoroughly mixed and moistened with water. The moist mass is pushed through a screen with a 1 mm mesh size, dried at about 45° C. and the granules are then passed through the same screen. After the magnesium stearate has been mixed in, convex tablet cores with a diameter of 11 mm are compressed in a tablet-making machine. The tablet cores thus produced are coated in known manner with a covering consisting essentially of sugar and talc. The finished coated tablets are polished with wax.

| D) | Capsules | per capsule |
|---|---|---|
| | Ambroxol | 250 mg |
| | Corn starch | 268.5 mg |
| | Magnesium stearate | 1.5 mg |

The ambroxol and corn starch are mixed and moistened with water. The moist mass is screened and dried. The dry granules are screened and mixed with magnesium stearate. The finished mixture is packed into size 1 hard gelatine capsules.

| E) | Parenteral solution | |
|---|---|---|
| | ambroxol | 500 mg |
| | citric acid monohydrate | 100 mg |
| | sodium hydroxide | 35 mg |
| | mannitol | 1500 mg |
| | water for inj. | 50 ml |

The ambroxol is dissolved in water at its own pH or optionally at pH 4.5 to 5.5 and mannitol is added to make it isotonic. The solution obtained is filtered free from pyrogens and the filtrate is transferred under aseptic conditions into injection bottles which are then sealed with rubber stoppers and autoclaved.

| F) | Suppositories | |
|---|---|---|
| | Ambroxol | 450 mg |
| | Solid fat | 1650 mg |

The hard fat is melted. At 40° C. the ambroxol is homogeneously dispersed. It is cooled to 38° C. and poured into slightly chilled suppository moulds.

| G) | Oral solution | |
|---|---|---|
| | ambroxol | 150 mg |
| | hydroxyethylcellulose | 50 mg |
| | sorbic acid | 5 mg |
| | sorbitol (70%) | 600 mg |
| | glycerol | 200 mg |
| | flavouring | 15 mg |
| | water ad | 10 ml |

Distilled water is heated to 70° C. Hydroxyethylcellulose is dissolved therein with stirring. After the sorbitol solution and glycerol have been added the mixture is cooled to ambient temperature. At ambient temperature sorbic acid, flavouring and ambroxol are added. To remove air from the suspension it is evacuated with stirring.

| H) | Ointment | | |
|---|---|---|---|
| | composition g/100 g ointment: | | |
| | ambroxol | 20 | g |
| | sodium disulphite | 0.1 | g |
| | cetylalcohol | 10 | g |
| | stearylalcohol | 10 | g |
| | white Vaseline | 5 | g |
| | perfume oil q.s. | | |
| | distilled water ad | 100 | g |

The ingredients are processed in the usual way to form an ointment.

What is claimed is:

1. A method for teating diseases or conditions which are based on a powerful activation of voltage-dependent sodium channels, which method comprises administering to a host suffering from such a disease a therapeutically effective amount of ambroxol or a pharmacologically acceptable salt thereof.

2. The method of claim 1 wherein the disease or condition is based on the activation of tetrodotoxin-resistant sodium channels.

3. The method of claim 1 wherein the disease or condition to be treated is chronic pain.

4. The method of claim 1 wherein the disease or condition to be treated is a cerebral excitotoxicity-induced disorder.

5. The method of claim 1 wherein the disease or condition to be treated is a cardiac arrhythmia.

6. The method of claim 1, 2, 3, 4 or 5 wherein there is further administered an active substance selected from the group consisting of the analgesics, anticonvulsants, neuroprotective substances and antiarrhythmics.

7. The method of claim 1, 2, 3, 4 or 5 wherein there is further administered an active substance selected from the group consisting of the opiates, non-steroidal analgesics, gabapentine and antidepressants and alpha-adrenergic agonists.
